(19) United States
(12) Patent Application Publication
(10) Pub. No.: US 2024/0161912 A1
(43) Pub. Date: May 16, 2024

(54) ARTIFICIAL INTELLIGENCE SYSTEM FOR FACILITATING ROUTING TO A PROVIDER

(71) Applicant: Helix Virtual Medicine, Inc., Boca Raton, FL (US)

(72) Inventors: ROBERT RODRIGUEZ, Hillsboro Beach, FL (US); ELIZABETH J DELONG, Port St. Lucie, FL (US)

(73) Assignee: Helix Virtual Medicine, Inc., Boca Raton, FL (US)

(21) Appl. No.: 18/508,073

(22) Filed: Nov. 13, 2023

Related U.S. Application Data
(60) Provisional application No. 63/424,629, filed on Nov. 11, 2022.

(51) Int. Cl. G16H 40/20

(52) U.S. Cl. CPC G16H 40/20

(57) ABSTRACT

A system for facilitating routing to a provider via artificial intelligence is provided. The system interacts with a user to obtain information from the user during an encounter. The information is provided to an artificial intelligence engine to determine a medical complaint of the user. The artificial intelligence engine proceeds to generate a list of providers matching criteria associated with the medical complaint, the information, or a combination thereof. Additional filters are applied to the list of providers to enhance correlation of the providers in the list for the individual. The artificial intelligence engine determines an optimal provider from the list based on the optimal provider having a greater correlation with the information, the medical complaint, or a combination thereof, than other providers in the list. The system presents the list of providers and digital links to enable selection of one or more providers to establish connections with the providers.

### CROSS REFERENCE TO RELATED APPLICATIONS

The present application claims priority to and the benefit of U.S. Provisional Patent Application No. 63/424,629, filed on Nov. 11, 2022, the entirety of which is hereby incorporated by reference. Additionally, U.S. patent application Ser. No. 18/381,103 and PCT/US23/35334, which were filed on Oct. 17, 2023, and are commonly owned, along with U.S. Provisional Application No. 63/416,753, are all hereby incorporated by reference in their entireties. Furthermore, U.S. patent application Ser. No. 18/497,709, which was filed on Oct. 30, 2023, along with U.S. Provisional Application No. 63/420,321 are also hereby incorporated by reference in their entireties.

### FIELD OF THE INVENTION

The present application relates to artificial intelligence technologies, automation technologies, sensor technologies, case management technologies, healthcare onboarding technologies, routing technologies, and, more particularly, to an artificial intelligence system and accompanying methods for facilitating routing to a provider.

### BACKGROUND

Currently, patients have a variety of ways in which they can connect with providers. For example, patients may go directly to a hospital, participate in patient intake, and get routed to the appropriate provider based on the symptoms being experienced by the patient, based on an existing relationship with a provider, or based on other criteria. As another more recent example, instead of going directly to a hospital, the patient may schedule a telemedicine or telehealth digital visit online by utilizing various computing devices to interact with and obtain a diagnosis from a provider remotely. Telehealth services often resolves the need for the increase in resources for patients who may not be able to access traditional healthcare services. Despite the convenience of telehealth services, certain orders and prescriptions require that the physician provider be located within the same state or territory as the patient. In addition, some insurance companies will only honor prescriptions placed by providers that are “in-network”. Often times, available telehealth providers may or may not meet such criteria.

Many traditional telehealth systems are requiring patients to pay for the service themselves and therefore the patients not able to use the insurance benefits they have available to them. In certain instances, existing telehealth platforms will present the patient with provider options with difficult to parse detail and then expect the patient to sort through and select the provider that most closely meets their needs. These provider details are often self-reported and are not updated on regular basis, which leads to patient frustration. Such frustrations are often exacerbated when the patient either cannot fill their prescriptions or cannot get reimbursement from their insurance.

Based on at least the foregoing, there remains room for substantial enhancements to existing technologies and processes and for the development of new technologies and processes to facilitate patient onboarding and outboarding and routing to providers. For example, current technologies may be improved and enhanced so as to provide for more effective patient intake and registration, improved medical appointment scheduling, enhanced provider recommendation capabilities, enhanced diagnostic capabilities, greater quality data, faster processing of patient data, improved medical record generation capabilities, and other enhancements. Such enhancements and improvements to methodologies and technologies may provide for increased standardization for patient onboarding and outboarding, improved patient compliance, reduced administrative work, enhanced patient-provider satisfaction, among other benefits.

### SUMMARY

A system and accompanying methods for providing and facilitating routing to a provider using artificial intelligence are disclosed. In particular, the system and methods provide a patient onboarding and outboarding platform incorporating algorithms that facilitate patient intake, generation of a physician-ready triage note, generation of a differential patient diagnosis, generation of a treatment plan and creation of orders prior to a physician examining a patient. In certain embodiments, the system and methods may include utilizing one or more artificial intelligence engines to interact with and obtain information from a user or individual, such as a patient. Such information, for example, may include, but is not limited to, a reason for the user's encounter, payment information, geolocation information, symptom information, demographic information, other types of information, or a combination thereof. In certain embodiments, the system and methods may analyze the information obtained from the user to facilitate a determination of a medical complaint associated with the user. In certain embodiments, the system and methods may utilize the artificial intelligence engines to generate a list of providers matching criteria associated with the determined medical complaint, the information obtained from the user, any other information, or a combination thereof.

In certain embodiments, the system and methods may identify an optimal provider from the list of providers. For example, the optimal provider may be a provider from the list of providers that has a greatest correlation with the criteria associated with the medical complaint, the information obtained from the user, any other information, or a combination thereof. In certain embodiments, the system and methods may present the list of providers and generate links that enable the selection of one or more providers to establish connections between the user and the one or more providers. Once one or more providers are selected from the list, the system and methods may establish the connections between the user and the one or more providers. In certain embodiments, the connections may comprise establishing communication between the user and a provider, initiating a telemedicine visit with the provider, scheduling an appointment with the provider, scheduling a lab test, any type of connection, or a combination thereof. In certain embodiments, the system and methods may then utilize information associated with the selection of the providers, connections, information associated with the user, determined medical complaints, any other information, or a combination thereof, to train artificial intelligence models supporting the functionality of the artificial intelligence engines of the system. In certain embodiments, the process may be repeated for each new encounter a user has with the system, for each new user of the system, and as often as desired. Based on at least the foregoing, the system and methods may optimize the routing of users to providers, while simultaneously reducing user frustration, reduce physician overload by connecting user needs to the specific providers that are able to address those needs, provide greater standards of care compliance through mutually shared user and provider data, and provide greater efficiencies by connecting the users to the appropriate provider of care.

In certain embodiments, a system for facilitating routing to a provider using artificial intelligence is provided. The system may include a memory that stores instructions and a processor that executes the instructions to perform various operations of the system. The system may perform an operation that includes registering, such as via an interface of the system, an individual with the system. For example, the registering may include, but is not limited to, obtaining demographic information, identification information, psychographic information, insurance information, payment information, any type of information, or a combination thereof. Additionally, in certain embodiments, the registration may include obtaining consents from the individual to perform medical procedures, consents to be examined, consents to perform any other type of activity that may be consented to, or a combination thereof. The system may then perform an operation that includes interacting with the individual to obtain information from the individual, such as information relating to a medical condition or complaint. In certain embodiments, the interacting may be conducted by utilizing an artificial intelligence engine. The system may perform an operation that includes determining, by utilizing the artificial intelligence engine and based on the information, the medical complaint associated with the individual. The medical complaint may be identified based on the information having a correlation with medical complaint information that is utilized to train the artificial intelligence engine.

In certain embodiments, the system may perform an operation that includes generating, by utilizing the artificial intelligence engine, a list of providers matching criteria associated with the medical complaint, the information, or a combination thereof. In certain embodiments, the system may perform an operation that includes identifying, by utilizing the artificial intelligence engine, an optimal provider from the list of providers. In certain embodiments, the optimal provider may have a greater correlation with the criteria than other providers in the list of providers. In certain embodiments, the system may include presenting, via the interface, the list of providers and a digital link enabling selection of the optimal provider to establish a connection with the optimal provider. In certain embodiments, digital links to select other providers from the list may also be provided.

In certain embodiments, a method for facilitating routing to a provider using artificial intelligence is disclosed. The method may include a memory that stores instructions and a processor that executes the instructions to perform the functionality of the method. In particular, the method may include registering, via an interface of a system and during an encounter, an individual with the system. Additionally, the method may include interacting, by utilizing an artificial intelligence engine, with the individual to obtain information from the individual. The method may also include determining, by utilizing the artificial intelligence engine and based on the information, a medical complaint associated with the individual. In certain embodiments, the medical complaint may be identified based on the information having a correlation with medical complaint information utilized to train the artificial intelligence engine. The method may also include generating, by utilizing the artificial intelligence engine, a list of providers matching criteria associated with the medical complaint, the information, or a combination thereof. Furthermore, the method may include identifying, by utilizing the artificial intelligence engine, an optimal provider from the list of providers. In certain embodiments, the optimal provider has a greater correlation with the criteria than other providers in the list of providers. Moreover, the method may include presenting, via the interface, the list of providers and a digital link enabling selection of the optimal provider to establish a connection with the optimal provider.

According to further embodiments, a computer-readable device comprising instructions, which, when loaded and executed by a processor cause the processor to perform operations, the operations comprising: registering, via an interface of a system and during an encounter, an individual with the system; interacting, by utilizing an artificial intelligence engine, with the individual to obtain information from the individual; determining, by utilizing the artificial intelligence engine and based on the information, a medical complaint associated with the individual, wherein the medical complaint is identified based on the information having a correlation with medical complaint information utilized to train the artificial intelligence engine; generating, by utilizing the artificial intelligence engine, a list of providers matching criteria associated with the medical complaint, the information, or a combination thereof; identifying, by utilizing the artificial intelligence engine, an optimal provider from the list of providers, wherein the optimal provider has a greater correlation with the criteria than other providers in the list of providers; and providing, via the interface, the list of providers and a digital link enabling selection of the optimal provider to establish a connection with the optimal provider.

These and other features of the systems and methods for facilitating routing to a provider using artificial intelligence are described in the following detailed description, drawings, and appended claims.

### BRIEF DESCRIPTION OF THE DRAWINGS

FIG. 1 is a schematic diagram of a system for facilitating routing to a provider by utilizing artificial intelligence according to embodiments of the present disclosure.

FIG. 2 illustrates an exemplary process flow for use with the system of FIG. 1 that enables patient registration, generates digital records including plans for the patient, facilitates patient encounters with a provider, and facilitates digital record editing according to embodiments of the present disclosure

FIG. 3 illustrates an exemplary process flow for use with the system of FIG. 1 that facilitates updates to digital records of a patient, validates plans for patients, and facilitates medical billing according to embodiments of the present disclosure.

FIG. 4 illustrates an exemplary process flow for use with the system of FIG. 1 that facilitates patient registration, obtaining information from a patient, determining a list of providers matching criteria, applying filters, and presenting provider options for selection according to embodiments of the present disclosure.

FIG. 5 illustrates an exemplary process flow for use with the system of FIG. 1 that facilitates billing and compliance screening according to embodiments of the present disclosure.

FIG. 6 is a flow diagram illustrating a sample method for facilitating routing to a provider using artificial intelligence according to embodiments of the present disclosure.

FIG. 7 is a schematic diagram of a machine in the form of a computer system within which a set of instructions, when executed, may cause the machine to facilitate the routing to a provider according to embodiments of the present disclosure.

### DETAILED DESCRIPTION

A system 100 and accompanying methods for providing and facilitating routing to a provider using artificial intelligence are disclosed. In particular, the system 100 and methods provide a patient onboarding and outboarding platform incorporating algorithms that facilitate patient intake, generation of a physician-ready triage note, generation of a differential patient diagnosis, generation of a treatment plan and creation of orders prior to a physician examining a patient. In certain embodiments, the system 100 and methods may include utilizing one or more artificial intelligence engines to interact with and obtain information from a user or individual, such as a patient. Such information, for example, may include, but is not limited to, a reason for the user's encounter, payment information, geolocation information, symptom information, demographic information, identification information, other types of information, or a combination thereof. In certain embodiments, the system 100 and methods may analyze the information obtained from the user to facilitate a determination of a medical complaint associated with the user. In certain embodiments, the system 100 and methods may utilize the artificial intelligence engines to generate a list of providers matching criteria associated with the determined medical complaint, the information obtained from the user, any other information, or a combination thereof.

In certain embodiments, the system 100 and methods may identify an optimal provider from the list of providers. In certain embodiments, for example, the optimal provider may be a provider from the list of providers that has a greatest correlation with the criteria associated with the medical complaint, the information obtained from the user, any other information, or a combination thereof. In certain embodiments, the system 100 and methods may present the list of providers and generate links that enable the selection of one or more providers to establish connections between the user and the one or more providers. Once one or more providers are selected from the list, the system and methods may establish the connections between the user and the one or more providers. In certain embodiments, the connections may comprise establishing communication between the user and a provider, initiating a telemedicine visit with the provider, scheduling an appointment with the provider, scheduling a lab test, any type of connection, or a combination thereof. In certain embodiments, the system and methods may then utilize information associated with the selection of the providers, connections, information associated with the user, determined medical complaints, any other information, or a combination thereof, to train artificial intelligence models supporting the functionality of the artificial intelligence engines of the system. In certain embodiments, the process may be repeated for each new encounter a user has with the system, for each new user of the system, and as often as desired. Based on at least the foregoing, the system and methods may optimize the routing of users to providers, while simultaneously reducing user frustration, reduce physician overload by connecting user needs to the specific providers that are able to address those needs, provide greater standards of care compliance through mutually shared user and provider data, and provide greater efficiencies by connecting the users to the appropriate provider of care.

In certain embodiments, the system 100 and methods may include determining, based on analyzing insurance information for the user (e.g., patient), whether the insurance information indicates that the insurance coverage requires an in-state provider for the user. In certain embodiments, if the insurance coverage requires an in-state provider, the artificial intelligence engine may filter out any providers in a pool of providers that are out of state. In certain embodiments, the artificial intelligence engine may determine if there are other criteria that may be utilized to filter the list of providers down for enhancing generation of provider options for routing the user. In certain embodiments, the criteria may be determined by the artificial intelligence engine itself, such as by analyzing the characteristics of the user and the information provided by the user to the system 100. In certain embodiments, the criteria may be supplied directly by the user, such as by receiving inputs from the user indicating the criteria. For example, the criteria specified by a user may include, but are not limited to, a preference for a provider gender, a preference for a provider location, a preference for a provider language, a preference for an in-network provider, any other preference, a preference for an insurance accepted by the provider, any other preferences, or a combination thereof. In certain embodiments, the criteria may be supplied by any number of providers, which may be received as inputs from the providers. For example, the criteria provided by the providers may include, but are not limited to, a preference for a type of insurance carrier for users, a preference for a type of medical complaint, a preference for a type of payment, a preference for a type of medical history, a preference for a location for the individual, any other preferences, or a combination thereof.

In certain embodiments, the artificial intelligence engine may analyze the criteria and generate a list of providers that may be provided as options for the user. In certain embodiments, based on the level of correlation with matching the criteria, the providers may in the list may be sorted. For example, a provider having a highest correlation with the criteria may be set as first in the list and the lowest correlation may be last in the list. In certain embodiments, only providers having a threshold level of correlation with the criteria may be provided in the list. In certain embodiments, the system 100 may be configured to generate interactable digital links for each provider and render the list of providers with the links to the user, such as via an application supporting the functionality of the system 100. The links may be utilized to establish a connection with a provider. For example, the links may be to initiate a chat session, initiate a video teleconference (e.g. telemedicine session), exchange media content, provide information about a provider and their capabilities, schedule appointments, set preferences for providers, any other interactions, or a combination thereof.

In certain embodiments, along with the links, the system 100 and methods may include presenting information indicating current availability of each provider, a state of license of the provider, whether the license is current and active, the provider's name, the provider's location, the type of provider, the specialties, capabilities, and/or treatments provided by the provider, reviews and/or ratings of the provider, a real-time indication of a level of correlation with criteria (e.g. changes as criteria changes and/or as information associated with the user changes (e.g., location of the user changes or symptoms of the user changes), any other information, or a combination thereof. In certain embodiments, the system 100 and methods may include receiving a selection of one or more providers from the list, such as from a user device of the user. In certain embodiments, the system 100 and methods may include training the artificial intelligence engines based on the selections made by the user, the generated lists, the information obtained from the user, any other information utilized to facilitate generation of the lists, any other information, or a combination thereof. In certain embodiments, the list of providers may be adjusted in real-time as criteria changes, as information received from the user changes, as characteristics associated with providers change, or a combination thereof.

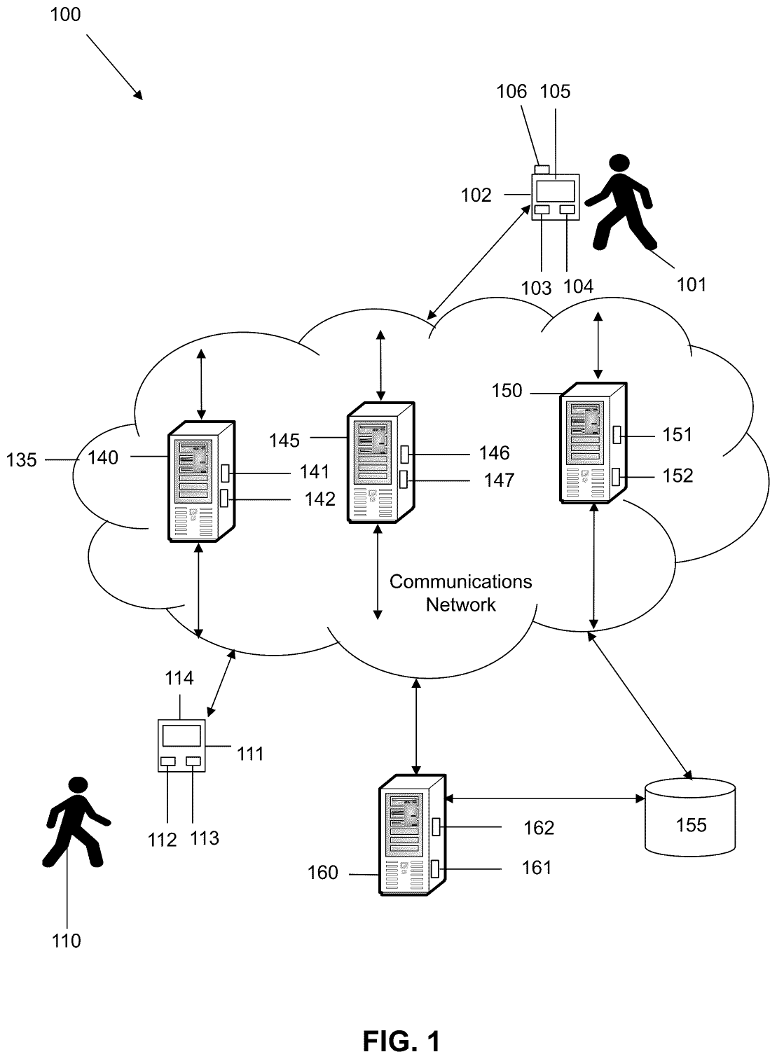

As shown in FIG. 1, a system for facilitating routing to a provider according to embodiments of the present disclosure is disclosed. Notably, the system 100 may be configured to support, but is not limited to supporting, healthcare systems, patient intake systems, patient digital records systems, patient routing systems, medical diagnosis systems, automation systems, data analytics systems and services, data collation and processing systems and services, artificial intelligence services and systems, machine learning services and systems, content delivery services, cloud computing services, satellite services, telephone services, voice-over-internet protocol services (VoIP), software as a service (SaaS) applications, platform as a service (PaaS) applications, social media applications and services, operations management applications and services, productivity applications and services, mobile applications and services, and/or any other computing applications and services. Notably, the system 100 may include a first user 101, who may utilize a first user device 102 to access data, content, and services, or to perform a variety of other tasks and functions. As an example, the first user 101 may utilize first user device 102 to transmit signals to access various online services and content, such as those available on an internet, on other devices, and/or on various computing systems. As another example, the first user device 102 may be utilized to access an application, devices, and/or components of the system 100 that provide any or all of the operative functions of the system 100. For example, the first user 101 may utilize the first user device 102 to access an application having a user interface that enables the first user 101 to submit personal data into the system 100 to register the first user 101 with the system 100 for purposes of patient intake and examination by a provider (e.g., second user 110 or a hospital system at which the second user 110 works at). As another example, the first user 101 may utilized the first user device 102 to interact with the application so that the application may determine a provider matching criteria associated with the first user 101. In certain embodiments, the first user 101 may be a bystander, any type of person, a robot, a humanoid, a program, a computer, any type of user, or a combination thereof, that may be located in a particular environment.

In certain embodiments, the first user 101 may be a person that may be experiencing a medical condition, may be seeking to having a health checkup, may be seeking a medical treatment, or a combination thereof. For example, the first user 101 may be a patient of a physician (e.g., the second user 110). In certain embodiments, the first user 101 may be a person that may be having a follow-up medical visit, may be visiting a referral provider (e.g., a specialist), or a combination thereof. In certain embodiments, the first user device 102 may be utilized by the first user to interact with the system 100, other users of the system 100, or a combination thereof. In certain embodiments, the first user device 102 may include a memory 103 that includes instructions, and a processor 104 that executes the instructions from the memory 103 to perform the various operations that are performed by the first user device 102. In certain embodiments, the processor 104 may be hardware, software, or a combination thereof. The first user device 102 may also include an interface 105 (e.g. screen, monitor, graphical user interface, etc.) that may enable the first user 101 to interact with various applications executing on the first user device 102 and to interact with the system 100. In certain embodiments, the first user device 102 may be and/or may include a computer, any type of sensor, a laptop, a set-top-box, a tablet device, a phablet, a server, a mobile device, a smartphone, a smart watch, and/or any other type of computing device. Illustratively, the first user device 102 is shown as a smartphone device in FIG. 1. In certain embodiments, the first user device 102 may be utilized by the first user 101 to control and/or provide some or all of the operative functionality of the system 100.

In addition to using first user device 102, the first user 101 may also utilize and/or have access to additional user devices. As with first user device 102, the first user 101 may utilize the additional user devices to transmit signals to access various online services and content. The additional user devices may include memories that include instructions, and processors that executes the instructions from the memories to perform the various operations that are performed by the additional user devices. In certain embodiments, the processors of the additional user devices may be hardware, software, or a combination thereof. The additional user devices may also include interfaces that may enable the first user 101 to interact with various applications executing on the additional user devices and to interact with the system 100. In certain embodiments, the first user device 102 and/or the additional user devices may be and/or may include a computer, any type of sensor, a laptop, a set-top-box, a tablet device, a phablet, a server, a mobile device, a smartphone, a smart watch, and/or any other type of computing device, and/or any combination thereof. Sensors may include, but are not limited to, cameras, location sensors, accelerometers, gyroscopes, motion sensors, acoustic/audio sensors, pressure sensors, temperature sensors, light sensors, heart-rate sensors, blood pressure sensors, sweat detection sensors, breath-detection sensors, stress-detection sensors, any type of health sensor, humidity sensors, any type of sensors, or a combination thereof. In certain embodiments, the first user device 102 may include a transceiver

The first user device 102 and/or additional user devices may belong to and/or form a communications network. In certain embodiments, the communications network may be a local, mesh, or other network that enables and/or facilitates various aspects of the functionality of the system 100. In certain embodiments, the communications network may be formed between the first user device 102 and additional user devices through the use of any type of wireless or other protocol and/or technology. For example, user devices may communicate with one another in the communications network by utilizing any protocol and/or wireless technology, satellite, fiber, or any combination thereof. Notably, the communications network may be configured to communicatively link with and/or communicate with any other network of the system 100 and/or outside the system 100.

In certain embodiments, the first user device 102 and additional user devices belonging to the communications network may share and exchange data with each other via the communications network. For example, the user devices may share information associated with a user (e.g., patient) with each other, information associated with digital records generated and/or maintained by the system 100, information relating to lab results, information relating to medical or physical examinations conducted by a physician on a user, information relating to the various components of the user devices, information associated with images and/or content accessed by a user of the user devices, information identifying the locations of the user devices, information indicating the types of sensors that are contained in and/or on the user devices, information identifying the applications being utilized on the user devices, information identifying how the user devices are being utilized by a user, information identifying user profiles for users of the user devices, information identifying device profiles for the user devices, information identifying the number of devices in the communications network, information identifying devices being added to or removed from the communications network, any other information, or any combination thereof.

In addition to the first user 101, the system 100 may also include a second user 110. The second user 110 may be a person that may facilitate treatment of the first user 101. For example, in certain embodiments, the second user 110 may be a physician, nurse, technician, intake professional, pharmacist, or other individual that work at a hospital, medical practice, any other location, or a combination thereof. In certain embodiments, the second user device 111 may be utilized by the second user 110 to transmit signals to request various types of content, services, and data provided by and/or accessible by communications network 135 or any other network in the system 100. In certain embodiments, the second user device 111 may be utilized by the second user 110 to view patient data, generate plans for patients, edit plants for patients, provide instructions for patients, confirm the content of digital records generated by the system 100, perform any operative functionality of the system 100, or a combination thereof. In further embodiments, the second user 110 may be a robot, a computer, a vehicle (e.g. semi or fully-automated vehicle), a humanoid, an animal, any type of user, or any combination thereof. The second user device 111 may include a memory 112 that includes instructions, and a processor 113 that executes the instructions from the memory 112 to perform the various operations that are performed by the second user device 111. In certain embodiments, the processor 113 may be hardware, software, or a combination thereof. The second user device 111 may also include an interface 114 (e.g. screen, monitor, graphical user interface, etc.) that may enable the first user 101 to interact with various applications executing on the second user device 111 and, in certain embodiments, to interact with the system 100. In certain embodiments, the second user device 111 may be a computer, a laptop, a set-top-box, a tablet device, a phablet, a server, a mobile device, a smartphone, a smart watch, and/or any other type of computing device. Illustratively, the second user device 111 is shown as a mobile device in FIG. 1. In certain embodiments, the second user device 111 may also include sensors, such as, but are not limited to, cameras, accelerometers, gyroscopes, location sensors (e.g. global positioning sensors), audio sensors, motion sensors, pressure sensors, temperature sensors, light sensors, heart-rate sensors, blood pressure sensors, sweat detection sensors, breath-detection sensors, stress-detection sensors, any type of health sensor, humidity sensors, any type of sensors, or a combination thereof.

In certain embodiments, the first user device 102, the additional user devices, and/or the second user device 111 may have any number of software applications and/or application services stored and/or accessible thereon. For example, the first user device 102, the additional user devices, and/or the second user device 111 may include applications for controlling and/or accessing the operative features and functionality of the system 100, applications for controlling and/or accessing any device of the system 100, healthcare applications, patient record management applications, patient record generating applications, medical billing applications, interactive social media applications, biometric applications, cloud-based applications, VoIP applications, other types of phone-based applications, product-ordering applications, business applications, e-commerce applications, media streaming applications, content-based applications, media-editing applications, database applications, gaming applications, internet-based applications, browser applications, mobile applications, service-based applications, productivity applications, video applications, music applications, social media applications, any other type of applications, any types of application services, or a combination thereof. In certain embodiments, the software applications may support the functionality provided by the system 100 and methods described in the present disclosure. In certain embodiments, the software applications and services may include one or more graphical user interfaces so as to enable the first and/or potentially second users 101, 110 to readily interact with the software applications. The software applications and services may also be utilized by the first and/or potentially second users 101, 110 to interact with any device in the system 100, any network in the system 100, or any combination thereof. In certain embodiments, the first user device 102, the additional user devices, and/or potentially the second user device 111 may include associated telephone numbers, device identities, or any other identifiers to uniquely identify the first user device 102, the additional user devices, and/or the second user device 111.

The system 100 may also include a communications network 135. The communications network 135 may be under the control of a service provider, any designated user, a computer, another network, or a combination thereof. The communications network 135 of the system 100 may be configured to link each of the devices in the system 100 to one another. For example, the communications network 135 may be utilized by the first user device 102 to connect with other devices within or outside communications network 135. Additionally, the communications network 135 may be configured to transmit, generate, and receive any information and data traversing the system 100. In certain embodiments, the communications network 135 may include any number of servers, databases, or other componentry. The communications network 135 may also include and be connected to a mesh network, a local network, a cloud-computing network, an IMS network, a VoIP network, a security network, a VoLTE network, a wireless network, an Ethernet network, a satellite network, a broadband network, a cellular network, a private network, a cable network, the Internet, an internet protocol network, MPLS network, a content distribution network, any network, or any combination thereof. Illustratively, servers 140, 145, and 150 are shown as being included within communications network 135. In certain embodiments, the communications network 135 may be part of a single autonomous system that is located in a particular geographic region or be part of multiple autonomous systems that span several geographic regions.

Notably, the functionality of the system 100 may be supported and executed by using any combination of the servers 140, 145, 150, and 160. The servers 140, 145, and 150 may reside in communications network 135, however, in certain embodiments, the servers 140, 145, 150 may reside outside communications network 135. The servers 140, 145, and 150 may provide and serve as a server service that performs the various operations and functions provided by the system 100. In certain embodiments, the server 140 may include a memory 141 that includes instructions, and a processor 142 that executes the instructions from the memory 141 to perform various operations that are performed by the server 140. The processor 142 may be hardware, software, or a combination thereof. Similarly, the server 145 may include a memory 146 that includes instructions, and a processor 147 that executes the instructions from the memory 146 to perform the various operations that are performed by the server 145. Furthermore, the server 150 may include a memory 151 that includes instructions, and a processor 152 that executes the instructions from the memory 151 to perform the various operations that are performed by the server 150. In certain embodiments, the servers 140, 145, 150, and 160 may be network servers, routers, gateways, switches, media distribution hubs, signal transfer points, service control points, service switching points, firewalls, routers, edge devices, nodes, computers, mobile devices, or any other suitable computing device, or any combination thereof. In certain embodiments, the servers 140, 145, 150 may be communicatively linked to the communications network 135, any network, any device in the system 100, or any combination thereof.

The database 155 of the system 100 may be utilized to store and relay information that traverses the system 100, cache content that traverses the system 100, store data about each of the devices in the system 100 and perform any other typical functions of a database. In certain embodiments, the database 155 may be connected to or reside within the communications network 135, any other network, or a combination thereof. In certain embodiments, the database 155 may serve as a central repository for any information associated with any of the devices and information associated with the system 100. Furthermore, the database 155 may include a processor and memory or may be connected to a processor and memory to perform the various operation associated with the database 155. In certain embodiments, the database 155 may be connected to the servers 140, 145, 150, 160, the first user device 102, the second user device 111, the additional user devices, any devices in the system 100, any process of the system 100, any program of the system 100, any other device, any network, or any combination thereof.

The database 155 may also store information and metadata obtained from the system 100, store metadata and other information associated with the first and second users 101, 110, store registration information, store generated lists of providers, store optimal providers, store filters and/or criteria utilized to filter the lists of providers, store information indicating matching of criteria associated with users/patients with providers, store provide license information, store rendering insurance numbers, store billing national provider identifiers, store information for referral partners, store artificial intelligence models utilized in the system 100, store sensor data and/or content obtained from a patient and/or user device, store predictions made by the system 100 and/or artificial intelligence models, store confidence scores relating to predictions made, store threshold values for confidence scores, store responses outputted and/or facilitated by the system 100, store information associated with anything determined or detected via the system 100, store information and/or content utilized to train the artificial intelligence models, store information associated with behaviors and/or actions conducted by individuals, store user profiles associated with the first and second users 101, 110, store device profiles associated with any device in the system 100, store communications traversing the system 100, store user preferences, store information associated with any device or signal in the system 100, store information relating to patterns of usage relating to the user devices 102, 111, store any information obtained from any of the networks in the system 100, store historical data associated with the first and second users 101, 110, store device characteristics, store information relating to any devices associated with the first and second users 101, 110, store information associated with the communications network 135, store any information generated and/or processed by the system 100, store any of the information disclosed for any of the operations and functions disclosed for the system 100 herewith, store any information traversing the system 100, or any combination thereof.

In certain embodiments, the database 155 may be configured to store information supplied by the patient to register with the system 100, information associated with patient encounters with the system and/or providers, information associated with the patient's health status, digital records, lab results, information associated with surgical procedures to be performed or already performed on the patient, plans generated by the system 100, edits to plans generated by the system 100, medical billing information, insurance information, information relating to patient visits and medical conditions, information associated with medical complaints made by a patient or determined by the system 100, information associated with recommendations for treatments to be done for the patient, information associated with medication to be taken by the patient, information identifying standing order protocols, information identifying the patient and/or physician, any other information of the system 100, or a combination thereof. Furthermore, the database 155 may be configured to process queries sent to it by any device in the system 100.

In certain embodiments, the system 100 may incorporate the use of any number of artificial intelligence engines, such as, but not limited to, a triage artificial intelligence engine 204, a physician assessment engine 208, other artificial intelligence engines, or a combination thereof. In certain embodiments, the triage artificial intelligence engine 204 may contain some or all of the functionality of the physician assessment engine 208 and/or other artificial intelligence engines, or vice versa. In certain embodiments, the system 100 may include one or more artificial intelligence models supporting the functionality of the system 100, a triage artificial intelligence engine 204 and/or the physician assessment engine 208. In certain embodiments, an artificial intelligence model may be a file, program, module, and/or process that may be trained by the system 100 (or other system) to recognize certain patterns, diagnoses, health conditions, diseases, behaviors, and/or content. For example, the artificial intelligence model(s) may be trained to determine medical complaints (i.e., what the user is currently experiencing from a health standpoint), detect specific types of diseases afflicting a user of the system 100, generate a plan to treat detection diseases and/or conditions, generate assessment codes (e.g., CPT codes or other codes) that may be utilized for billing purposes or for obtaining prescriptions.

In certain embodiments, the artificial intelligence model(s) may be trained to determine a list of providers that match criteria associated with and/or for a patient, determine optimal providers having a strongest correlation with criteria associated with and/or for a patient, determine providers for patients based on criteria specific by providers and/or by the patients, or any combination thereof. In certain embodiments, the artificial intelligence model may be, may include, and/or may utilize a Deep Convolutional Neural Network, a one-dimensional convolutional neural network, a two-dimensional convolutional neural network, a Long Short-Term Memory network, any type of machine learning system, any type of artificial intelligence system, or a combination thereof. Additionally, in certain embodiments, the artificial intelligence model may incorporate the use of any type of artificial intelligence and/or machine learning algorithms to facilitate the operation of the artificial intelligence model(s).

The system 100 may train the artificial intelligence model(s) to reason and learn from data fed into the system 100 so that the model(s) may generate and/or facilitate the generation of predictions about new data and information that is fed into the system 100 for analysis. For example, the system 100 may train an artificial intelligence model using various types of data, information, and/or content, such as, but not limited to, images, video content, audio content, text content, augmented reality content, virtual reality content, information relating to patterns, information relating to behaviors, information relating to characteristics of providers and/or referral providers, information relating to characteristics of users, information relating to environments, sensor data, information from medical libraries, information associated with diseases or medical conditions, any data associated with the foregoing, any type of data, or a combination thereof. In certain embodiments, the content and/or data utilized to train the artificial intelligence model may be utilized to correlate and/or associate user-provided information to specific detectable medical conditions, medical treatment plans, assessments, and the like. As additional data and/or content is fed into the model(s) over time, the model's ability to recognize medical complaints, generate plans, and determine assessments will improve and be more finely tuned.

In certain embodiments, the triage artificial intelligence engine 204 may be configured to engage and/or interact with a user (e.g., patient) to facilitate identification of a medical complaint associated with the user and/or to generate a list of providers matching criteria associated with the user. For example, the triage artificial intelligence engine may be configured to transmit messages to the user via an application accessible by the first user device 102 requesting that the user provide information associated with what the user is feeling, what symptoms the user has, any other information, or a combination thereof. Based on the interactions with the user, the triage artificial intelligence engine 204 may determine the medical complaint of the user (e.g., what the user is complaining about from a health standpoint). Additionally, in certain embodiments, the triage artificial intelligence engine 204 may be configured to facilitate the automatic generation of digital record that may include a ready-for-execution medical note, such as a S.O.A.P. note. The note, for example, may include a predicted assessment (e.g., diagnosis or medical condition) for the user, a treatment plan for treating the diagnosis or medical condition, subject information associated with the user, and objective data associated with the user (e.g., lab results, measurements, vital signs, etc.). In certain embodiments, assessment codes (CPT codes or other codes that may be used for billing purposes or insurance purposes) may also be predicted by the triage artificial intelligence system.

In certain embodiments, the physician assessment engine 208, may be configured to facilitate confirmation of the information in the auto-generated digital record, such as by providing the digital record to a physician for further review. In certain embodiments, the physician assessment engine 208 may also be configured to analyze the digital record and/or information associated with the user to determine whether to provide the digital record to a physician for further review or to finalize the digital record directly, such as if standing order protocols associated with the medical complaint and/or predicted assessment for the user exist. In certain embodiments, the physician assessment engine 208 may also be utilized to determine whether the user needs a face-to-face encounter with the physician, whether an in-person encounter is required, whether testing is to be performed on the user, whether a procedure is to be performed with respect to the user, whether the user should go directly to a hospital or other facility within a vicinity of the location of the user, along with other functional described in the present disclosure. The physician assessment engine 208 may be configured to perform its operative functionality by utilizing any number of artificial intelligence models and functionality.

In certain embodiments, the triage artificial intelligence engine 204, the physician assessment engine 208, another artificial intelligence engine, or a combination thereof, may be utilized to determine where to route a user (e.g., patient), such as when the patient has an encounter with the system 100. In certain embodiments, for example, the triage artificial intelligence engine 204 may interact with the user via an application executing on the first user device 102 and may pose questions to the user. In certain embodiments, the questions may be tailored to the user based on the information input by the user during the user's registration with the system 100. The user may provide responses, such as via speech, text, video, or other methodologies for inputting responses, such as by utilizing the first user device 102. The responses may then be utilized by the triage artificial intelligence engine 204 to determine a medical complaint for the user and characteristics associated with the user. In certain embodiments, for example, the triage artificial intelligence engine 204 may determine the medical complaint based on the information from the user having a correlation with medical complaint information of the system 100 utilized to train the triage artificial intelligence engine 204.

Based on the medical compliant, criteria specified by the user, criteria specified by providers, and/or criteria selected by the triage artificial intelligence engine 204, the triage artificial intelligence engine 204 may analyze provider information stored in the system 100 (e.g., in the database 155) and generate a list of providers matching the criteria. In certain embodiments, matching the criteria may include having a threshold level of correlation and/or overlap between the criteria be utilized and the characteristics of the providers. In certain embodiments, the triage artificial intelligence engine 204 may identify an optimal provider of the providers in the list based on the optimal provider having characteristics having a greatest correlation with the criteria when compared with other providers in the list. In certain embodiments, the triage artificial intelligence engine 204 may generate digital links, interfaces, digital buttons, interactable objects, and/or other features, which may be provided by the application for interaction by the user. The user, for example, may select one or more providers and/or establish a direct connection with the provider. In certain embodiments, the system 100 may enable the user to set an appointment with a provider, speak with the provider, text the provider, teleconference with the provider, connect to a device of the provider, obtain additional information from the provider, or a combination thereof.

In certain embodiments, the triage artificial intelligence engine 204, the physician assessment engine 208, and/or other artificial intelligence engines may be trained with any of the data generated, accessed, and/or modified by the system 100. In certain embodiments, the user may submit reviews of a provider or feedback relating to the provider, which may be utilized to modify how the provider is ranked in a future list of providers generated by the system 100, identify whether the level of criteria matching predicted by the artificial intelligence engine was accurate, and adjust predictions for matching criteria for future encounters with the user and/or other users of the system 100.

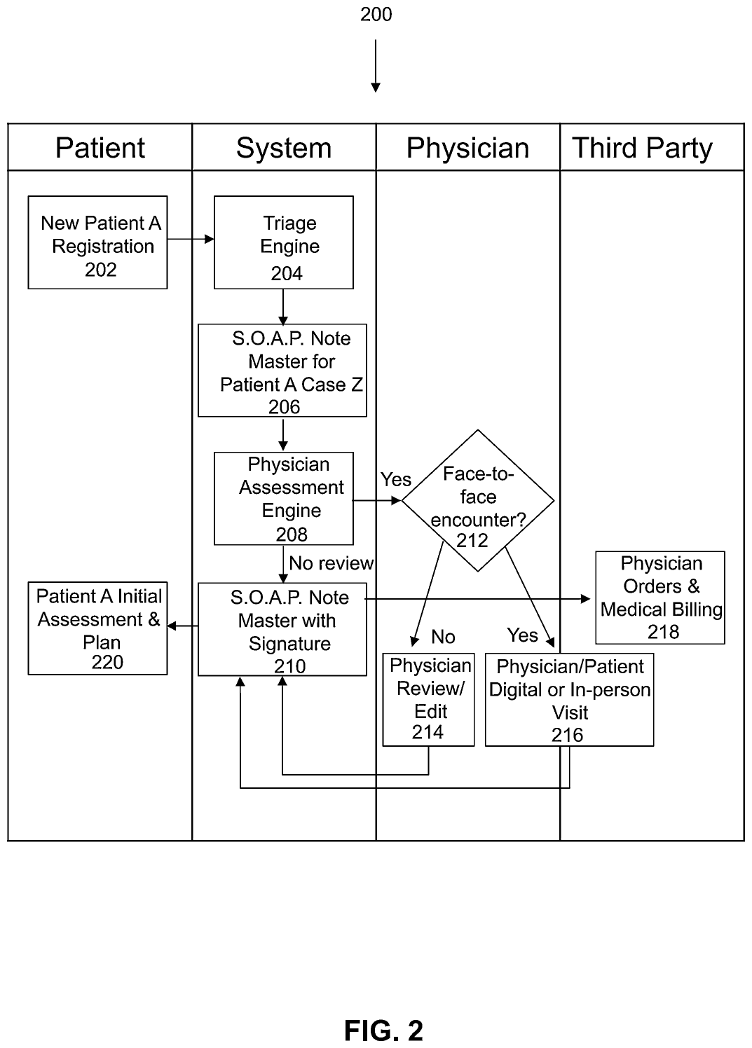

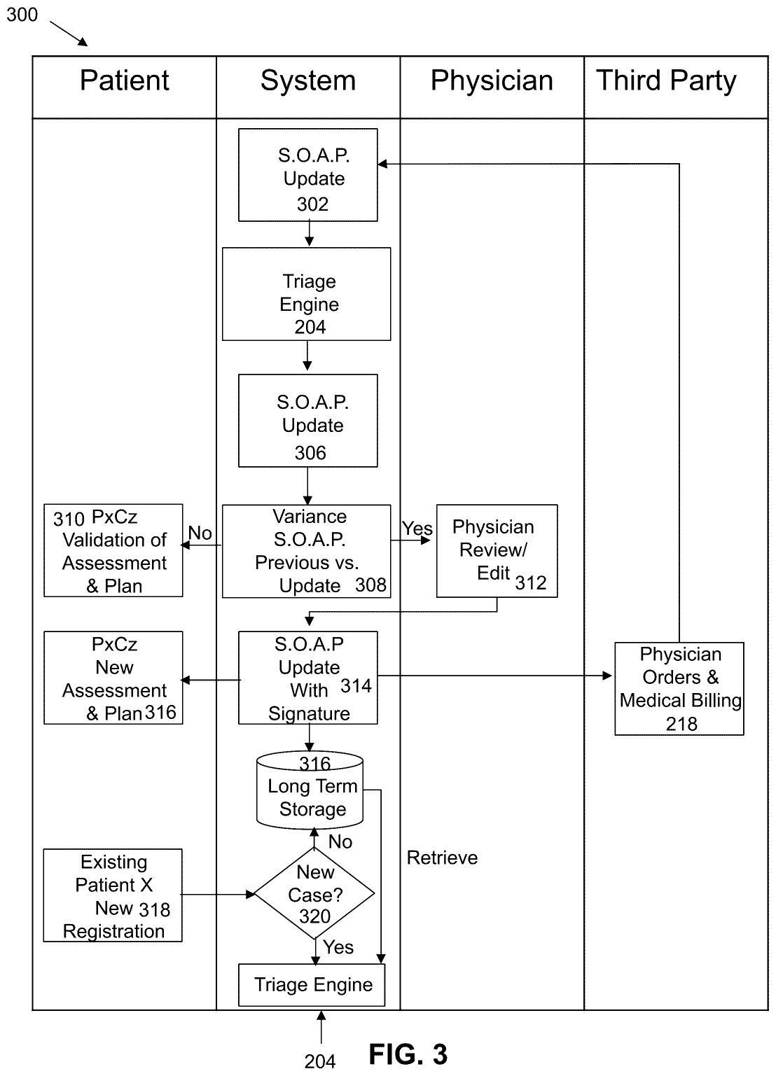

Operatively, the system 100 may operate and/or execute the functionality as described and illustrated in FIGS. 2, 3, 4, 5, or as otherwise described herein. FIG. 2 illustrates an exemplary process flow for use with the system 100 that enables patient registration, generates digital records including plans for the patient, facilitates patient encounters with a provider, and facilitates digital record editing according to embodiments of the present disclosure. FIG. 3 illustrates an exemplary process flow for use with the system 100 that facilitates updates to digital records of a patient, validates plans for patients, and facilitates medical billing according to embodiments of the present disclosure. Referring initially to FIG. 2, the process flow 200 may include, at 202, registering a patient with the system 100. For example, the user (e.g., first user 101) may access an application supporting the functionality of the system 100 by utilizing first user device 102. The application functionality and features may be accessible by rendered graphical user interface that may be displayed on an interface of the first user device 102. The user may register may inputting demographic information, psychographic information, identity information, location information, physiological information, any other information, or a combination thereof. In certain embodiments, during the registration process, the user may also be provided with consent forms that may required the user's consent or authorization before the user may have an examination, procedure, or treatment.

At 204, the flow 200 may include utilizing the triage artificial intelligence engine 204 to interact with the user to extra further information from the user to determine the user's medical complaint. For example, the triage artificial intelligence engine 204 may pose questions to the user, the responses to which may be utilized to determine the medical complaint. Such questions may include questions relating to the symptoms that the user is experiencing, a history of such symptoms, the foods that the user ate, the medications that the user is taking, whether others in the user's vicinity are experiencing symptoms, any other questions, or a combination thereof. In certain embodiments, the triage artificial intelligence engine 204 may be configured to interact with the user via voice-based communications, text-based communications, video-related communications, augmented reality based communications, virtual reality based communications, any other communication technology, or a combination thereof. Once the user provides the information in response to the interactions with the engine, the process flow 200 may proceed to 206. At 206, the flow 200 may include auto-generating a digital record for the user. As indicated here, the digital record may include a digital S.O.A.P. note including subjective data associated with the user, objective data associated with the user, an assessment (e.g., diagnosis) for the user, a plan for treatment of the condition associated with the diagnosis, or a combination thereof. In certain embodiments, the note may include all the elements to treat and bill the user (e.g., Patient A, Case Z (“PaCz”)). In certain embodiments, the plan generated by the engine 204 may include information relating to labs, imaging, medication, medical equipment, and specialist treatment needed for the user.

The process flow 200 may include providing the digital record to the physician assessment engine 208 for review and processing. In certain embodiments, the physician assessment engine 208 may be configured to filter the digital record for the user to establish workflow priorities. For example, the physician assessment engine 208 may be configured to (1) determine whether the digital record is to be completed and signed as-is; (2) determining whether the physician needs to visually review the digital record prior to completion and signing; (3) whether the user requires a telemedicine or in-person visit with the physician; and (4) whether the user needs to go to the hospital or other treatment facility immediately. In certain embodiments, while generic standards of care may be embedded in the triage artificial intelligence engine 204, rendering provider/physician specific protocols to create proper workflow priority may be required. For example, the physician assessment engine 208 may determine that the digital record does not need further review because standing order protocols that dictate the protocol for the specific diagnosis for the user already exist. In such a scenario, the flow 200 may proceed to 210 where the digital record may be finalized and marked complete by the system 100. The digital record (or at least the digital S.O.A.P. note) may be signed by the physician (e.g. via digital signature, authentication (e.g., biometric), or other technique). In certain embodiments, the finalized digital record may include all elements to treat and bill the user. The plan of the digital record may identify labs, imaging, medications, medical equipment, and specialist treatment needed for the user. The digital record may be utilized to inform and educate the user as well as provide instruction to third parties on the method of care needed for the user. The digital record may be fully billable for all insurance payors. Once finalized, the flow 200 may proceed to 220 where the user's initial assessment and plan may be completed and provided to the user, provided to medical billing systems for medical billing at 218, or a combination thereof. At 218, receiving electronic, written, or verbal objective information back from third parties may be key to proper and effective case management.

If, however, the physician assessment engine 208 determines that the digital record does need further review, the flow 200 may proceed to 212, where the physician assessment engine 208 may determine whether the user requires a face-to-face encounter with the physician. If the user is determined not to require a face-to-face encounter with the physician, the flow 200 may proceed to 214, where the flow 200 may include having the physician review and/or edit the digital record based on the information that the system 100 currently has. The digital record then may be finalized at 210 and signed off by the physician so that the flow 200 may proceed to 220, where the plan and assessment may be completed. If, however, at 212, the physician assessment engine 208 determines that the user requires a face-to-face encounter, the flow 200 may proceed to 216, where the user may either have a telemedicine encounter with the physician or may have an in-person visit at a facility that the physician works at. Based on the encounter, the physician may have additional information for the digital record and may either confirm the information in the digital record or edit/modify the information contained therein. Then, as with the other scenarios, the assessment and plan may be completed at 220 and provided to the user and to medical billing at 218.

Referring now also to FIG. 3, an exemplary process flow 300 for use with the system of FIG. 1 that facilitates updates to digital records of a patient, validates plans for patients, and facilitates medical billing according to embodiments of the present disclosure. At 302, updates to the digital record of the user needed to re-run the triage artificial intelligence engine 204 to generate a derivative digital record including a derivative note may be conducted. The update process may be iterative and may start with a master digital record plus new objective data filtered through the triage artificial intelligence engine 204 to produce a derivative digital record. Once filtered through the triage artificial intelligence engine 204, the flow 300 may conduct further updates to the digital record at 306. At 308, the flow may include conducting a variance analysis of the original digital record in comparison to the updated version of the digital record. In certain embodiments, the variance analysis of the digital record to the derivative digital record may be utilized to identify gaps and/or treatment plan changes between digital records. In certain embodiments, at 308, the physician may be enabled to addresses possible errors, new data, and make changes as needed and to publish a revised digital record including a revised S.O.A.P. note. In certain embodiments, the updates to the digital record may be looped back to the physician assessment engine 208 through certain rendering physicians may wish to manually review any changes to the original assessment and plan.

If there is no variance between the original digital record and the derivative/updated digital record, the flow 300 may proceed to 310 and validate the assessment and plan from the original digital record. If, however, there is a variance or discrepancy at 308, the flow 300 may proceed to 312. At 312, the physician may review to confirm the variance or edit/correct potential errors or inaccuracies. At 314, the flow 300 may include finalizing the digital record and obtaining the signature from the physician to complete the digital record. As a result, the flow 300 may proceed to 316, which results in the generation of a new assessment and plan for the user. Additionally, the digital record may be provided to a medical billing system at 218 for further review. The completed and signed digital record may be stored in long term storage at 316 and may be provided to the user to information and educate the user, as well as provide instructions to third parties on the method of care for the user.

At 318, the same user may conduct a new registration with the system 100 for another encounter with the system 100. At 320, the system 100 may determine if the encounter is a new case. If not, the flow 300 may proceed to obtain the digital records stored at 316 and provide them to the user and/or the physician for review. If, however, it is a new case/encounter for a new ailment, the flow 300 may proceed to utilizing the artificial intelligence triage engine 204, which may retrieve the saved digital record and then update the digital record for the new encounter and proceed through the steps of flows 200 and 300 as needed.

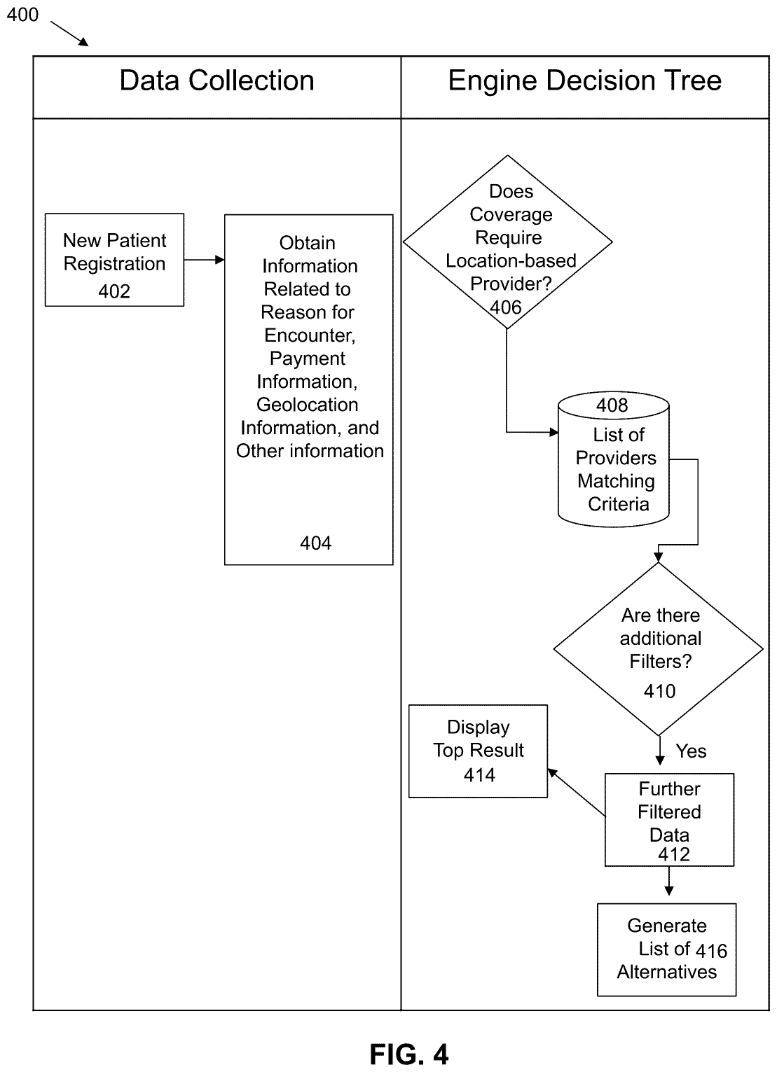

Referring now also to FIG. 4, an exemplary process flow 400 for use with the system 100 of FIG. 1 that facilitates patient registration, obtaining information from a patient, determining a list of providers matching criteria, applying filters, and presenting provider options for selection according to embodiments of the present disclosure is shown. At 402, a patient (e.g., first user 101) may register with the system 100. In certain embodiments, for example, the user may register via a user interface of an application accessible by the first user device 102. In certain embodiments, during the registration process and/or after the registration process, the user, at 404, may input a reason for the encounter (e.g., an identification of symptoms, how the user feels, monthly check-up, or other reason), payment information, insurance information, geolocation information, demographic information, any type of information, or a combination thereof. In certain embodiments, the inputted information may be conducted via text message, instant message, speech, typed text, audio-visual inputs, haptic inputs, symbol inputs, emoji inputs, any type of inputs, or a combination thereof.

In certain embodiments, an artificial intelligence engine (e.g., triage artificial intelligence engine 204) analyze and compare the inputted information to information associated with and/or contained within the system 100. In certain embodiments, the information associated with and/or contained with the system 100 may be information utilized to train the artificial intelligence engine to detect patterns, images, correlations, presence of information, matches with providers, any type of detectable information, or a combination thereof. At 406, the artificial intelligence engine may analyze the insurance coverage information provided by the user either during the registration process or after and may determine whether the coverage is location-specific. For example, artificial intelligence engine may determine if the insurance for the user requires the use of an in-state provider (i.e., a provider located in the same state as the user and/or the state that the policy is associated with). In certain embodiments, the requirement of using an in-state provider for the insurance may be an initial criteria that may be utilized to determine which providers would be appropriate for the user. At step 408, the artificial intelligence engine may, based on the determination that the insurance requires an in-state provider, filter out all out-of-state providers from the possible provider options to be provided in a generated list of provider options to the user.

At 410, once the out-of-state providers are filtered out of the list of providers, the artificial intelligence engine may determine whether there are any other criteria or filters that are to be applied in order to finely tune the list for the user. For example, other criteria may have been specified by the user, other criteria may have been specified by one or more providers, other criteria may be suggested, recommended, and/or utilized by the artificial intelligence engine, or a combination thereof. For example, criteria from the user may include, but is not limited to, a preference for a provider gender, a preference for a provider location, a preference for a provider language, a preference for an in-network provider, a preference for a certain amount of experience, a preference for various board certifications, any type of preference, or a combination thereof. As another example, criteria from the provider(s) may include a preference for a type of insurance carrier, a preference for a type of medical complaint, a preference for a type of payment, a preference for a type of medical history, a preference for a location for the individual, any other preferences, or a combination thereof. As a further example, criteria suggested, predicted, and/or selected by the artificial intelligence engine and/or system 100 itself may be criteria that the artificial intelligence engine and/or system 100 determines would be likely or have a threshold probability of being criteria that the provider and/or user would specify.

In certain embodiments, the artificial intelligence engine and/or system 100 may be configured to analyze information provided by the user and/or information associated with the user and compare it to information previously obtained from the user during a prior encounter and/or to information associated with other users. For example, if another user has a threshold similarity (e.g., lives in the same geographic location or within range of a geographic location, has a similar medical complaint, has similar symptoms, has similar type of upbringing and/or family, has had a similar medical history, and/or other similarities) with the user, criteria selected by the other user may be indicative of and/or have a correlation with criteria that may be selected or desired by the user during the current encounter. A similar process may be conducted for providers. In certain embodiments, the artificial intelligence engine may prompt the user regarding suggested or recommended criteria. For example, based on the analysis, the artificial intelligence engine may prompt the user “do you wish to see a female physician who speaks Spanish?” Once the additional criteria/filters are determined and/or predicted, the artificial intelligence engine may utilize the additional criteria/filters to further filter the list of potential provider options for the user, at 412. At 412, a filtered list of providers may be generated.

At 414, the artificial intelligence engine may be configured to determine and present the optimal provider from the list of provider options. In certain embodiments, the optimal provider may be the provider having a greatest correlation with the criteria. In certain embodiments, the correlation may be expressed as a percentage, as a visual representation, an audio representation, and/or other representation via the user interface of the application. In certain embodiments, the artificial intelligence engine and system 100 may generate digital links to establish communications with each provider and visual representations of the providers that may be rendered on a user interface of an application supporting the functionality of the system 100. At 416, the artificial intelligence engine may determine and present the other providers in the filtered list of providers to the user, such as via the interface. The user may then select one or more providers to schedule an appointment, communicate with the provider, establishing a connection with the provider, participate in a telemedicine visit, or a combination thereof. In certain embodiments, the selections made by the users may be utilized to train the artificial intelligence engine to present providers for subsequent encounters in different orders in the list for the same user or for another user having a correlation with the user. For example, the artificial intelligence engine may push up other providers in the list if they have just as good or better reviews or criteria in common as a selected provider, but for some reason (e.g., unrelated to performance) are not regularly selected by users. The process 400 may be repeated for each encounter, for each user of the system 100, as often as desired, as criteria changes in real-time, as interactions occur between a selected provider and the user, or a combination thereof.

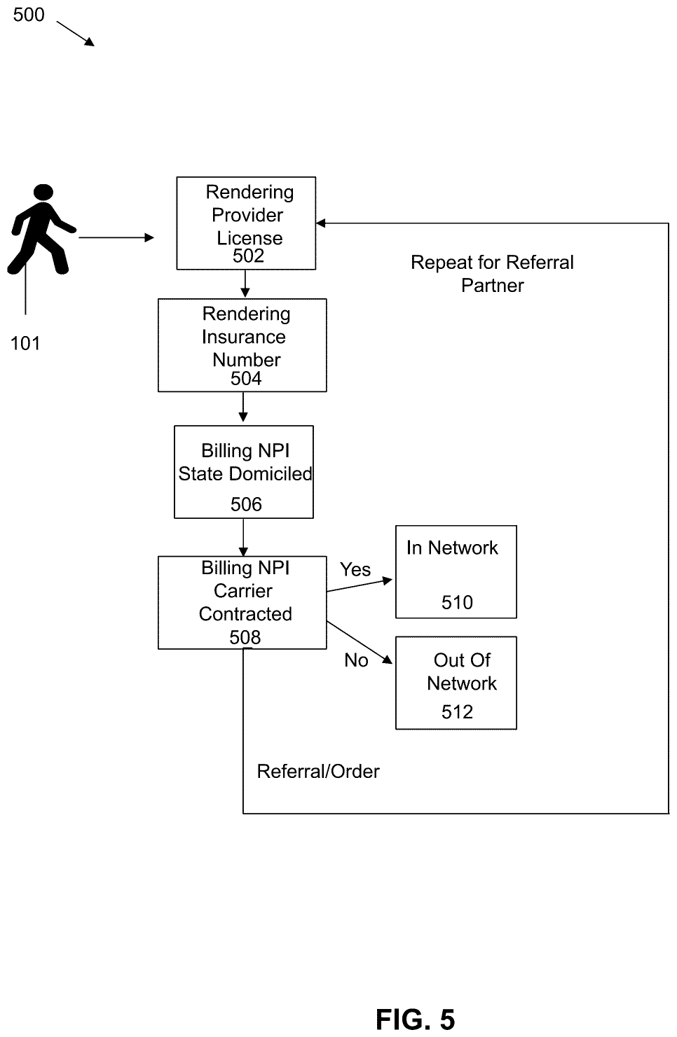

Referring now also to FIG. 5, FIG. 5 illustrates an exemplary process flow 500 for use with the system 100 of FIG. 1 that facilitates billing and compliance screening according to embodiments of the present disclosure. In certain embodiments, the process flow 500 may be utilized to eliminate issues that may arise when a provider in a hospital is in contract with an insurer, but the hospital or other facility is not. For example, the process flow 500 may be utilized to reduce issues associated with licensees (e.g., providers and hospitals or other facilities) when they collaborate on patient care, but the patient is exposed to both in-network (i.e., favorable to the patient) and out-of-network billing (i.e., unfavorable). In certain embodiments, the process flow 500 may be utilized to layer in legal compliance in the telemedicine context of corporate medicine and the legality of billing national provider identifier (NPI) being able to bill in the state that the patient is located in. In certain embodiments, the process flow 500 may be combined with any of the other process flows described herein.

At 502, the system 100 may confirm a rendering provider license, such as by comparing license information provided by the provider against against a database 155 that stores licenses of providers. For example, the rendering provider may be a provider selected by the user during process flow 400. At 504, the process flow 500 may include determining the rendering insurance number (e.g., rendering state medicated number). At 506, the process flow 500 may include confirming the billing NPI state of domicile. At 508, the process flow may determine the carrier contracted by the billing NPI. At 510, if the billing NPI is carrier contracted (e.g., matches insurance of patient), the system 100 may determine that the encounter or service provided by the provider is in-network. At 512, however, if the billing NPI is not carrier contracted (e.g., does not match insurance of the patient), the system 100 may determine that the encounter or services provided by the provider is out-of-network. Based on a visit, a provider may issue an order and/or referral to another provider and the processing 500 may be repeated accordingly. Based on the determinations and processes of process flow 500, the system 100 may utilize the information to generate higher quality lists for process flow 400. For example, the providers and/or facilities recommended in a list by the artificial intelligence engine may ensure that the facility (e.g., hospital) and the provider presented as an option are both in-network.

Notably, as shown in FIG. 1, the system 100 may perform any of the operative functions disclosed herein by utilizing the processing capabilities of server 160, the storage capacity of the database 155, or any other component of the system 100 to perform the operative functions disclosed herein. The server 160 may include one or more processors 162 that may be configured to process any of the various functions of the system 100. The processors 162 may be software, hardware, or a combination of hardware and software. Additionally, the server 160 may also include a memory 161, which stores instructions that the processors 162 may execute to perform various operations of the system 100. For example, the server 160 may assist in processing loads handled by the various devices in the system 100, such as, but not limited to, registering a user with the system 100; determining the user's medical complaint by utilizing the triage artificial intelligence engine 204 (or other artificial intelligence engine); generating digital records (e.g., S.O.A.P. notes), generating plans for a user based on analyzing user data, lab results, and other information; facilitating operative functionality of the physician assessment engine 208; updating digital records of a user; facilitating medical billing; interacting with users to obtain information; generating a list of providers matching criteria associated with a medical complaint, a user, other information, or a combination thereof; identifying optimal providers for the user; displaying possible provider options with digital links to establish a connection with one or more providers; training artificial intelligence models based on information obtained from a user, determinations made by the artificial intelligence engines, and/or selections made by a user (e.g., selecting one or more providers); and performing any other operations conducted in the system 100 or otherwise. In one embodiment, multiple servers 160 may be utilized to process the functions of the system 100. The server 160 and other devices in the system 100, may utilize the database 155 for storing data about the devices in the system 100 or any other information that is associated with the system 100. In one embodiment, multiple databases 155 may be utilized to store data in the system 100.

Although FIGS. 1-7 illustrate specific example configurations of the various components of the system 100, the system 100 may include any configuration of the components, which may include using a greater or lesser number of the components. For example, the system 100 is illustratively shown as including a first user device 102, a second user device 111, a communications network 135, a server 140, a server 145, a server 150, a server 160, and a database 155. However, the system 100 may include multiple first user devices 102, multiple second user devices 111, multiple communications networks 135, multiple servers 140, multiple servers 145, multiple servers 150, multiple servers 160, multiple databases 155, or any number of any of the other components inside or outside the system 100. Furthermore, in certain embodiments, substantial portions of the functionality and operations of the system 100 may be performed by other networks and systems that may be connected to system 100.

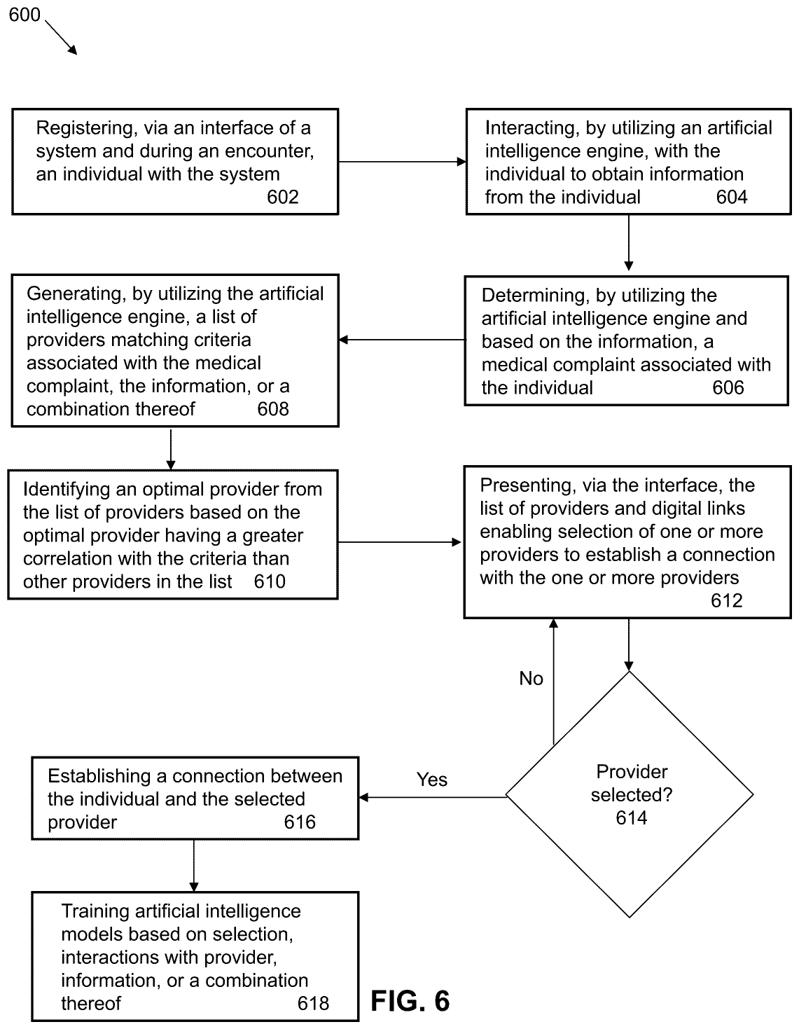

Notably, the system 100 may execute and/or conduct the functionality as described in the method(s) that follow. As shown in FIG. 6, an exemplary method 600 for providing an artificial intelligence system for facilitating routing to a provider is schematically illustrated. The method 600 and/or functionality and features supporting the method 600 may be conducted via an application of the system 100, devices of the system 100, processes of the system 100, any component of the system 100, or a combination thereof. The method 600 may include steps for obtaining data associated with a user (e.g., a patient), loading the data into artificial intelligence models for analysis, using the artificial intelligence models to compare the data to data utilized to train the artificial intelligence models, determining if the data associated with the user correlates and/or matches with data used to train the artificial intelligence models, generating predictions relating to a medical complaint of the user, generating lists of providers matching criteria associated with the medical complaint, matching the information associated with the user, or a combination thereof, and presenting digital links enabling selection of one or more providers to establish a connection with the one or more providers.

At step 602, the method 600 may include receiving information from a user (e.g., first user 101) to facilitate registration of the user with the system 100. For example, the user may input the information via a user interface of an application supporting the functionality of the system 100, such as via a first user device 102. The user may input information such as, but not limited to, demographic information, psychographic information, physiological information, payment information, health insurance information, any other information, or a combination thereof. Such information may include, but is not limited to, name, age, residence, current location, race, ethnicity, height, weight, eye color, skin color, body type, blood type, education level, income level, job title, credit card numbers, banking information, health insurance provider information (e.g., group number, individual number, etc.), mental state information, any other information, or a combination thereof. Additionally, in certain embodiments, the user may sign or otherwise consent (e.g., verbal authorization, biometric authorization, etc.) to consent forms that may be utilized to obtain consent from the user to receive treatment, examinations, and the like, such as from a physician (e.g., second user 110). In certain embodiments, the system 100 may also enable the user to create login credentials, which the user may utilized to authenticate into the application to access the system 100 and the user's information and/or digital records. In certain embodiments, the receiving of the information to register the user with the system 100 may be performed and/or facilitated by utilizing the first user 101, the second user 110 and/or by utilizing the first user device 102, the second user device 111, the server 140, the server 145, the server 150, the server 160, the communications network 135, any component of the system 100, any combination thereof, or by utilizing any other appropriate program, network, system, or device.

At step 604, the method 600 may include interacting with the user by utilizing a triage artificial intelligence engine (or other artificial intelligence engine) of the system 100. In certain embodiments, the triage artificial intelligence engine may be configured to engage with the user, such as via the application supporting the functionality of the system 100. In certain embodiments, the triage artificial intelligence engine may interact with the user via voice, text, instant messaging, chat, any other digital interaction technology, or a combination thereof. For example, the triage artificial intelligence engine may ask voice-based or text-based questions to the user to facilitate identification of a medical complaint of the user based on the responses to the questions. The user, for example, may respond with an identification of symptoms that the user is experiencing, medical history information, the length of time of the symptoms, the intensity of the symptoms, any other information associated with a medical complaint, or a combination thereof. In certain embodiments, the information may be input by the user into the application and may include triage information. In certain embodiments, the interacting may be performed by the system 100 through a variety of sensors. For example, the system 100 may utilize any number and/or combination of sensors to interact with and/or obtain information associated with and/or from the user. In certain embodiments, the system 100 can activate cameras, temperature sensors, pressure sensors, audio sensors, humidity sensors, motion sensors, light sensors, heart rate sensors, scanners (e.g., body scanners), breath-detection sensors, stress-detection sensors, any type of health sensor, any type of other sensor, or a combination thereof. In certain embodiments, the cameras and/or sensors can capture and/or measure sensor data and/or content to supplement and/or verify any information the user provides to the system 100. At step 606, the method 600 may include having the triage artificial intelligence system analyze the information to identify and/or determine the medical complaint that the user is experiencing. In certain embodiments, the medical complaint may be determined by comparing the information obtained from the user to data utilized to train artificial intelligence models supporting the functionality of the triage artificial intelligence engine. For example, image content taken of the user that is captured by a camera and sensor data obtained from sensors in a vicinity of the user can be analyzed by the triage artificial intelligence engine, which may have been trained on images and/or sensor data of and/or associated with various medical complaints, diseases, medical conditions, and the like.

In certain embodiments, the information from the user may be loaded into an artificial intelligence model(s) for analysis. In certain embodiments, artificial intelligence model(s) may be a file, program, module, and/or process that may be trained by the system 100 (or other system described herein) to recognize certain patterns, diagnoses, symptoms, behaviors, and/or content. For example, the artificial intelligence model(s) may be trained by the system 100 to detect specific types of objects, activity, occurrences, actions, motion, speed, and/or anything of interest. In certain embodiments, the artificial intelligence model may be, may include, and/or may utilize a Deep Convolutional Neural Network, a one-dimensional convolutional neural network, a two-dimensional convolutional neural network, a Long Short-Term Memory network, any type of machine learning system, any type of artificial intelligence system, or a combination thereof. In certain embodiments, the artificial intelligence model may incorporate the use of any type of artificial intelligence and/or machine learning algorithms to facilitate the operation of the artificial intelligence model(s). Notably, the system 100 may utilize any number of artificial intelligence models. The system 100 may train the artificial intelligence model(s) to reason and learn from data/information fed into the system 100 so that the model may generate and/or facilitate the generation of predictions about new data and information that is fed into the system 100 for analysis.

As an example, the artificial intelligence model(s) may be trained with data, such as, but not limited to, images, video content, audio content, text content, augmented reality content, virtual reality content, information relating to patterns, information relating to behaviors, information relating to characteristics of diseases, digital records containing patient data, sensor data (e.g., heart rate data, motion data, blood pressure data, oxygen data, blood glucose data, temperature data, etc.), any type of data, or a combination thereof. The data that is utilized to train the artificial intelligence model may be utilized by the artificial intelligence model to recognize diseases, medical conditions, psychological conditions, medical complaints (i.e., what the user is complaining about, such as foot pain, headache, stomach pain, etc.), or a combination thereof. For example, if the artificial intelligence model is trained with thousands of textual words that are known to be associated with the flu, the artificial intelligence model may learn that information that is fed into the model at a future time also are associated with the flu based on the future images and/or content having a correlation with the characteristics with any number of the textual words that are used to train the model. Similarly, if the artificial intelligence model is trained with image content and/or sensor data known to be associated with various medical complaints and/or conditions, the artificial intelligence model may learn that information that is fed into the model to determine various medical complaints and/or medical conditions of a user are associated with certain medial complaints and/or conditions that the model has been trained to detect and/or classify. As additional data and/or content is fed into the model(s) over time, the model's ability to recognize medica conditions, complaints, and/or diseases will improve and will be more finely tuned. In certain embodiments, interactions with the user and/or the loading of the data/information into the artificial intelligence model(s) for analysis may be performed and/or facilitated by utilizing the first user 101, the second user 110 and/or by utilizing the first user device 102, the second user device 111, the server 140, the server 145, the server 150, the server 160, the communications network 135, any component of the system 100, any combination thereof, or by utilizing any other appropriate program, network, system, or device.

At step 608, the method 600 may include generating, by utilizing the triage artificial intelligence engine, a list of providers matching criteria associated with the medical complaint, the information associated with the user, or a combination thereof. In certain embodiments, a provider may have matching criteria associated with the medical complaint, the information associated with the user, or a combination thereof, such as if the provider has capabilities to treat determined medical complaint and/or condition, if the provider has knowledge about the medical complaint and/or condition, if the provider has treated the medical complaint and/or condition in the past, if the provider has inventory of medicine or treatments to provide to the user, if the provider and/or devices of the provider are within a certain distance of the user and/or the user's device, if the provider accepts users into the provider's practice that have the medical complaint and/or condition, if the provider has equipment that is utilized to treat the medical complaint and/or condition, any other criteria, or a combination thereof. In certain embodiments, the generating of the list of providers may be performed and/or facilitated by utilizing the first user 101, the second user 110 and/or by utilizing the first user device 102, the second user device 111, the server 140, the server 145, the server 150, the server 160, the communications network 135, any component of the system 100, any combination thereof, or by utilizing any other appropriate program, network, system, or device. At step 610, the method 600 may include identifying the optimal provider from the list of providers based on the optimal provider having a greater correlation with the criteria than other providers in the list. In certain embodiments, the optimal provider can be the provider that has a greatest correlation with the criteria when compared to other providers having criteria matching the medical complaint, the information, and/or medical condition. For example, if one provider has a physician that is licensed to treat a certain medical complaint or condition, but another provider has a physician that is not only licensed to treat a certain medical compliant, but has done 1000 more cases than the physician of the other provider, the second provider can be optimal in comparison to the first provider. In certain embodiments, the optimal provider can be the provider that has a greatest number of matching criteria when compared with other providers. In certain embodiments, the criteria can be weighted according to importance and/or preference and the providers matching more criteria and/or higher weighted criteria can be more optimal than other providers. In certain embodiments, the identification of the optimal provider may be performed and/or facilitated by utilizing the first user 101, the second user 110 and/or by utilizing the first user device 102, the second user device 111, the server 140, the server 145, the server 150, the server 160, the communications network 135, any component of the system 100, any combination thereof, or by utilizing any other appropriate program, network, system, or device.

At step 612, the method 600 may include presenting, via the interface, the list of providers and digital links enabling selection of one or more providers to establish a connection with the one or more providers. In certain embodiments, the links can be utilized to establish a connection between a device of a provider and a device of the user, to establish a call (e.g., phone call or internet call) between the provider device and the user device, to establish a video conference between the provider and the user, to connect a device of the provider to any type of device of the user, any other type of connection, or a combination thereof. In certain embodiments, the presenting of the list and/or digital links may be performed and/or facilitated by utilizing the first user 101, the second user 110 and/or by utilizing the first user device 102, the second user device 111, the server 140, the server 145, the server 150, the server 160, the communications network 135, any component of the system 100, any combination thereof, or by utilizing any other appropriate program, network, system, or device. At step 614, the method 600 may include determining if a provider has been selected by the user/individual. If a selection has not been made, the method 600 may continue with step 612 until a selection is made. If, however, a selection of one or more providers is made at 614, the method 600 may proceed to step 616. At step 616, the method 600 may include establishing a connection between the user and the one or more selected providers. In certain embodiments, the establishing of the connection can be made directly by the device of the user and/or the device of the provider. For example, when a user clicks on a digital link to establish a connection with a provider, the clicking of the link can cause a device of a provider to establish a link to a user device so that that provider can be connected to the user via their corresponding devices. As another example, when a user clicks on the digital link, a call or conference can be initiated via the link to cause the connection to be established between a device of the provider and the device of the user. Once the provider and user are connected via the corresponding devices, the provider and user can conduct further interactions with each other to obtain additional information associated with the user, verify the medical complaint, provide treatment options, prescribe medication and/or treatments, and/or conduct any other activities to assist with the medical complaint and/or conditions associated with the user. In certain embodiments, when the provider and user are done interacting the connection can be terminated. In certain embodiments, the establishing of the connection may be performed and/or facilitated by utilizing the first user 101, the second user 110 and/or by utilizing the first user device 102, the second user device 111, the server 140, the server 145, the server 150, the server 160, the communications network 135, any component of the system 100, any combination thereof, or by utilizing any other appropriate program, network, system, or device.

At step 618, the method 600 may include training the artificial intelligence model(s) based on the selections of the providers made by the user, interactions between the user and the provider, the determined medical complaint, information provided by the user, predictions generated by the triage artificial intelligence engine, any information utilized by the system 100, or a combination thereof. In certain embodiments, the training may be configured to enhance predictions, deductions, reasoning, intelligence, correlations, outputs, analyses, and/or other capabilities of the artificial intelligence model(s). In certain embodiments, the training may be performed and/or facilitated by utilizing the first user 101, the second user 110 and/or by utilizing the first user device 102, the second user device 111, the server 140, the server 145, the server 150, the server 160, the communications network 135, any component of the system, any combination thereof, or by utilizing any other appropriate program, network, system, or device. Notably, the method 600 may further incorporate any of the features and functionality described for the system 100, any other method disclosed herein, or as otherwise described herein.

The systems and methods disclosed herein may include still further functionality and features. For example, the operative functions of the system 100 and method may be configured to execute on a special-purpose processor specifically configured to carry out the operations provided by the system 100 and method. Notably, the operative features and functionality provided by the system 100 and method may increase the efficiency of computing devices that are being utilized to facilitate the functionality provided by the system 100 and the various methods discloses herein. For example, by training the system 100 over time based on data and/or other information provided and/or generated in the system 100, a reduced amount of computer operations may need to be performed by the devices in the system 100 using the processors and memories of the system 100 than compared to traditional methodologies. In such a context, less processing power needs to be utilized because the processors and memories do not need to be dedicated for processing. As a result, there are substantial savings in the usage of computer resources by utilizing the software, techniques, and algorithms provided in the present disclosure. In certain embodiments, various operative functionality of the system 100 may be configured to execute on one or more graphics processors and/or application specific integrated processors.

Notably, in certain embodiments, various functions and features of the system 100 and methods may operate without any human intervention and may be conducted entirely by computing devices. In certain embodiments, for example, numerous computing devices may interact with devices of the system 100 to provide the functionality supported by the system 100. Additionally, in certain embodiments, the computing devices of the system 100 may operate continuously and without human intervention to reduce the possibility of errors being introduced into the system 100. In certain embodiments, the system 100 and methods may also provide effective computing resource management by utilizing the features and functions described in the present disclosure. For example, in certain embodiments, devices in the system 100 may transmit signals indicating that only a specific quantity of computer processor resources (e.g. processor clock cycles, processor speed, etc.) may be devoted to training the artificial intelligence model(s), comparing information obtained from a patient to information contained in and/or used by the artificial intelligence model(s), determining whether information correlates with information and/or content utilized to train an artificial intelligence model(s), generating predictions relating to plans, medical complaints, diagnoses, and/or other predictions, generating lists of providers matching criteria, identifying optimal providers, generating and presenting links to providers, establishing connections with providers, routing patients to providers, and/or performing any other operation conducted by the system 100, or any combination thereof. For example, the signal may indicate a number of processor cycles of a processor may be utilized to update and/or train an artificial intelligence model, and/or specify a selected amount of processing power that may be dedicated to generating or any of the operations performed by the system 100. In certain embodiments, a signal indicating the specific amount of computer processor resources or computer memory resources to be utilized for performing an operation of the system 100 may be transmitted from the first and/or second user devices 102, 111 to the various components of the system 100.

In certain embodiments, any device in the system 100 may transmit a signal to a memory device to cause the memory device to only dedicate a selected amount of memory resources to the various operations of the system 100. In certain embodiments, the system 100 and methods may also include transmitting signals to processors and memories to only perform the operative functions of the system 100 and methods at time periods when usage of processing resources and/or memory resources in the system 100 is at a selected value. In certain embodiments, the system 100 and methods may include transmitting signals to the memory devices utilized in the system 100, which indicate which specific sections of the memory should be utilized to store any of the data utilized or generated by the system 100. Notably, the signals transmitted to the processors and memories may be utilized to optimize the usage of computing resources while executing the operations conducted by the system 100. As a result, such functionality provides substantial operational efficiencies and improvements over existing technologies.

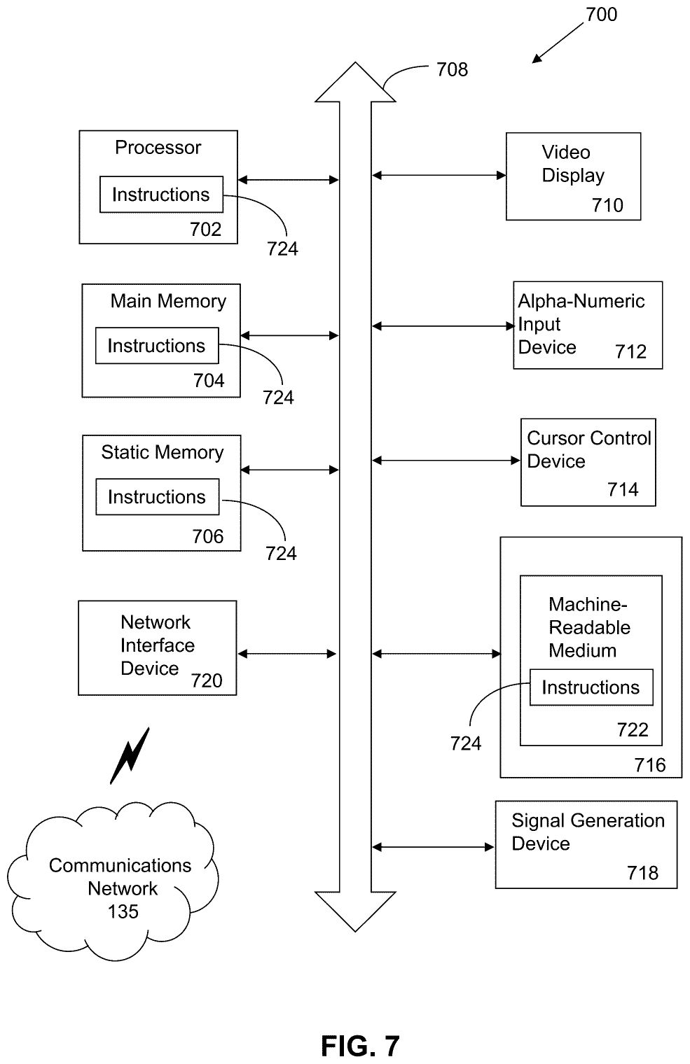

Referring now also to FIG. 7, at least a portion of the methodologies and techniques described with respect to the exemplary embodiments of the system 100 can incorporate a machine, such as, but not limited to, computer system 700, or other computing device within which a set of instructions, when executed, may cause the machine to perform any one or more of the methodologies or functions discussed above. The machine may be configured to facilitate various operations conducted by the system 100. For example, the machine may be configured to, but is not limited to, assist the system 100 by providing processing power to assist with processing loads experienced in the system 100, by providing storage capacity for storing instructions or data traversing the system 100, or by assisting with any other operations conducted by or within the system 100. As another example, the computer system 700 may assist with generating models associated with generating predictions relating to a diagnosis of a patient (e.g., first user 101), predictions relating to identification of a medical complaint of the patient, predictions relating to changes in a physical or mental condition of the patient over time, predictions relating to which provider(s) to route a patient to, predictions relating to referral providers that a patient may be referred to after connecting with an initial provider, any type of predictions generated by the system 100, or a combination thereof. As another example, the computer system 700 may assist with interacting with the patient (e.g., by transmitting questions to the first user 101, supporting a teleconference for a patient visit, any other interactions, or a combination thereof), registering the patient with the system 100, conducting medical billing, ensuring billing and compliance screening, determining whether a provider is licensed and/or is in-network or out-of-network, facilitating execution of physician orders for the patient, any other functionality provided by the system 100, or a combination thereof.

In certain embodiments, the machine may operate as a standalone device. In some embodiments, the machine may be connected (e.g., using communications network 135, another network, or a combination thereof) to and assist with operations performed by other machines and systems, such as, but not limited to, the first user device 102, the second user device 111, the server 140, the server 145, the server 150, the database 155, the server 160, any other system, program, and/or device, or any combination thereof. The machine may be connected with any component in the system 100. In a networked deployment, the machine may operate in the capacity of a server or a client user machine in a server-client user network environment, or as a peer machine in a peer-to-peer (or distributed) network environment. The machine may comprise a server computer, a client user computer, a personal computer (PC), a tablet PC, a laptop computer, a desktop computer, a control system, a network router, switch or bridge, or any machine capable of executing a set of instructions (sequential or otherwise) that specify actions to be taken by that machine. Further, while a single machine is illustrated, the term “machine” shall also be taken to include any collection of machines that individually or jointly execute a set (or multiple sets) of instructions to perform any one or more of the methodologies discussed herein.

The computer system 700 may include a processor 702 (e.g., a central processing unit (CPU), a graphics processing unit (GPU, or both), a main memory 704 and a static memory 706, which communicate with each other via a bus 708. The computer system 700 may further include a video display unit 710, which may be, but is not limited to, a liquid crystal display (LCD), a flat panel, a solid state display, or a cathode ray tube (CRT). The computer system 700 may include an input device 712, such as, but not limited to, a keyboard, a cursor control device 714, such as, but not limited to, a mouse, a disk drive unit 716, a signal generation device 718, such as, but not limited to, a speaker or remote control, and a network interface device 720.

In certain embodiments, the disk drive unit 716 may include a machine-readable medium 722 on which is stored one or more sets of instructions 724, such as, but not limited to, software embodying any one or more of the methodologies or functions described herein, including those methods illustrated above. The instructions 724 may also reside, completely or at least partially, within the main memory 704, the static memory 706, or within the processor 702, or a combination thereof, during execution thereof by the computer system 700. In certain embodiments, the main memory 704 and the processor 702 also may constitute machine-readable media.

Dedicated hardware implementations including, but not limited to, application specific integrated circuits, programmable logic arrays and other hardware devices can likewise be constructed to implement the methods described herein. Applications that may include the apparatus and systems of various embodiments broadly include a variety of electronic and computer systems. Some embodiments implement functions in two or more specific interconnected hardware modules or devices with related control and data signals communicated between and through the modules, or as portions of an application-specific integrated circuit. Thus, the example system is applicable to software, firmware, and hardware implementations.

In accordance with various embodiments of the present disclosure, the methods described herein are intended for operation as software programs running on a computer processor. Furthermore, software implementations can include, but not limited to, distributed processing or component/object distributed processing, parallel processing, or virtual machine processing can also be constructed to implement the methods described herein.

The present disclosure contemplates a machine-readable medium 722 containing instructions 724 so that a device connected to the communications network 135, another network, or a combination thereof, can send or receive voice, video or data, and communicate over the communications network 135, another network, or a combination thereof, using the instructions. The instructions 724 may further be transmitted or received over the communications network 135, another network, or a combination thereof, via the network interface device 720.

While the machine-readable medium 722 is shown in an example embodiment to be a single medium, the term “machine-readable medium” should be taken to include a single medium or multiple media (e.g., a centralized or distributed database, and/or associated caches and servers) that store the one or more sets of instructions. The term “machine-readable medium” shall also be taken to include any medium that is capable of storing, encoding or carrying a set of instructions for execution by the machine and that causes the machine to perform any one or more of the methodologies of the present disclosure.

The terms “machine-readable medium,” “machine-readable device,” or “computer-readable device” shall accordingly be taken to include, but not be limited to: memory devices, solid-state memories such as a memory card or other package that houses one or more read-only (non-volatile) memories, random access memories, or other re-writable (volatile) memories; magneto-optical or optical medium such as a disk or tape; or other self-contained information archive or set of archives is considered a distribution medium equivalent to a tangible storage medium. In certain embodiments, the “machine-readable medium,” “machine-readable device,” or “computer-readable device” may be non-transitory, and, in certain embodiments, may not include a wave or signal per se. Accordingly, the disclosure is considered to include any one or more of a machine-readable medium or a distribution medium, as listed herein and including art-recognized equivalents and successor media, in which the software implementations herein are stored.

The illustrations of arrangements described herein are intended to provide a general understanding of the structure of various embodiments, and they are not intended to serve as a complete description of all the elements and features of apparatus and systems that might make use of the structures described herein. Other arrangements may be utilized and derived therefrom, such that structural and logical substitutions and changes may be made without departing from the scope of this disclosure. Figures are also merely representational and may not be drawn to scale. Certain proportions thereof may be exaggerated, while others may be minimized. Accordingly, the specification and drawings are to be regarded in an illustrative rather than a restrictive sense.

Thus, although specific arrangements have been illustrated and described herein, it should be appreciated that any arrangement calculated to achieve the same purpose may be substituted for the specific arrangement shown. This disclosure is intended to cover any and all adaptations or variations of various embodiments and arrangements of the invention. Combinations of the above arrangements, and other arrangements not specifically described herein, will be apparent to those of skill in the art upon reviewing the above description. Therefore, it is intended that the disclosure is not limited to the particular arrangement(s) disclosed as the best mode contemplated for carrying out this invention, but that the invention will include all embodiments and arrangements falling within the scope of the appended claims.

The foregoing is provided for purposes of illustrating, explaining, and describing embodiments of this invention. Modifications and adaptations to these embodiments will be apparent to those skilled in the art and may be made without departing from the scope or spirit of this invention. Upon reviewing the aforementioned embodiments, it would be evident to an artisan with ordinary skill in the art that said embodiments can be modified, reduced, or enhanced without departing from the scope and spirit of the claims described below.

We claim:

1. A system, comprising:
a memory that stores instructions; and
a processor configured to execute the instructions to:
register, via an interface of the system and during an encounter, an individual with the system;
interact, by utilizing an artificial intelligence engine, with the individual to obtain information from the individual;
determine, by utilizing the artificial intelligence engine and based on the information, a medical complaint associated with the individual, wherein the medical complaint is identified based on the information having a correlation with medical complaint information utilized to train the artificial intelligence engine;
generate, by utilizing the artificial intelligence engine, a list of providers matching criteria associated with the medical complaint, the information, or a combination thereof;
identify, by utilizing the artificial intelligence engine, an optimal provider from the list of providers, wherein the optimal provider has a greater correlation with the criteria than other providers in the list of providers; and
present, via the interface, the list of providers and a digital link enabling selection of the optimal provider to establish a connection with the optimal provider.

2. The system of claim 1, wherein the information from the individual comprises a reason for the encounter, insurance information, payment information, demographic information, geolocation information, identification information, or a combination thereof.

3. The system of claim 2, wherein the processor is further configured to determine, based on analyzing the insurance information, whether the insurance information requires an in-state provider for the individual.

4. The system of claim 3, wherein the processor is further configured to filter each provider in the list of providers that is an out-of-state provider if the insurance information requires the in-state provider for the individual.

5. The system of claim 1, wherein the processor is further configured to receive a portion of the criteria from the individual, wherein the portion of the criteria comprises a preference for a provider gender, a preference for a provider location, a preference for a provider language, a preference for an in-network provider, or a combination thereof.

6. The system of claim 1, wherein the processor is further configured to receive a portion of the criteria from at least one provider from the list of providers, wherein the portion of the criteria comprises a preference for a type of insurance carrier, a preference for a type of medical complaint, a preference for a type of payment, a preference for a type of medical history, a preference for a location for the individual, or a combination thereof.

7. The system of claim 1, wherein the processor is further configured to sort the list of providers in accordance with a correlation of each provider in the list with the criteria.

8. The system of claim 1, wherein the processor is further configured to receive a selection of at least one provider from the list of providers.

9. The system of claim 8, wherein the processor is further configured to train the artificial intelligence model using training information including the selection, the list of providers matching the criteria, the information from the individual, or a combination thereof.

10. The system of claim 1, wherein the processor is further configured to generate digital links for each of the providers in the list of providers.

11. The system of claim 1, wherein the processor is further configured to obtain geolocation information of the information based on accessing a global positioning device of a device of the individual, connecting to receiver of the device of the individual, accessing a sensor of the device of the individual, or a combination thereof.

12. The system of claim 1, wherein the processor is further configured to display, via the interface, an amount of correlation with the criteria for each provider from the list of providers.

13. The system of claim 1, wherein the processor is further configured to receive additional criteria to further filter the list of providers.

14. A method, comprising:
registering, via an interface of a system and during an encounter, an individual with the system;
interacting, by utilizing an artificial intelligence engine, with the individual to obtain information from the individual;
determining, by utilizing the artificial intelligence engine and based on the information, a medical complaint associated with the individual, wherein the medical complaint is identified based on the information having a correlation with medical complaint information utilized to train the artificial intelligence engine;
generating, by utilizing the artificial intelligence engine and by utilizing instructions from a memory that are executed by a processor, a list of providers matching criteria associated with the medical complaint, the information, or a combination thereof;
identifying, by utilizing the artificial intelligence engine, an optimal provider from the list of providers, wherein the optimal provider has a greater correlation with the criteria than other providers in the list of providers; and
presenting, via the interface, the list of providers and a digital link enabling selection of the optimal provider to establish a connection with the optimal provider.

15. The method of claim 14, further comprising adjusting the list of providers in real-time as additional information from the individual, providers in the list of providers, or a combination thereof, arrive at the system.

16. The method of claim 14, further comprising determining whether each provider in the list of providers has a license in a state in which the medical complaint is to be treated, whether each provider is in-network or out-of-network, whether a referral provider has a license in the state in which the medical complaint is to be treated, whether a referral provider is in-network or out-of-network, or a combination thereof.

17. The method of claim 14, further comprising adjusting the list of providers as a location of the individual, a device of the individual, or a combination thereof, changes.

18. The method of claim 14, further comprising obtaining new information associated with a different encounter of the individual.

19. The method of claim 18, further comprising modifying the list of providers and identifying a new optimal provider based on the new information.

20. A non-transitory computer-readable device comprising instructions, which, when loaded and executed by a processor, cause the processor to perform operations, the operations comprising:
registering, via an interface of a system and during an encounter, an individual with the system;
interacting, by utilizing an artificial intelligence engine, with the individual to obtain information from the individual;
determining, by utilizing the artificial intelligence engine and based on the information, a medical complaint associated with the individual, wherein the medical complaint is identified based on the information having a correlation with medical complaint information utilized to train the artificial intelligence engine;
generating, by utilizing the artificial intelligence engine, a list of providers matching criteria associated with the medical complaint, the information, or a combination thereof;
identifying, by utilizing the artificial intelligence engine, an optimal provider from the list of providers, wherein the optimal provider has a greater correlation with the criteria than other providers in the list of providers; and
providing, via the interface, the list of providers and a digital link enabling selection of the optimal provider to establish a connection with the optimal provider.
